(19) United States
(12) Patent Application Publication
(10) Pub. No.: US 2007/0250142 A1
(43) Pub. Date: Oct. 25, 2007

(54) Atomic Layer Deposition Coatings for Implantable Medical Devices

(76) Inventors: Richard W.A. Francis, White Bear Lake, MN (US); Naim S. Istephanous, Roseville, MN (US); Eric H. Bonde, Victoria, MN (US); Michael A. Schugt, Saint Paul, MN (US); Richard J. O'Brien, Prior Lake, MN (US)

Correspondence Address: MEDTRONIC, INC., 710 MEDTRONIC PARKWAY NE, MINNEAPOLIS, MN, 55432-9924, US

(21) Appl. No.: 11/278,020

(22) Filed: Mar. 30, 2006

(51) Int. Cl. A61N 1/00

(52) U.S. Cl. 607/116

(57) ABSTRACT

Implantable medical devices with coatings formed by atomic layer deposition and methods of applying such coatings to implantable medical devices are disclosed. The medical devices may include electrical feedthroughs and media exposed integrated circuits and/or transducer systems, as well as others. The coatings may improve, among other things, hermeticity, biocompatibility, biostability, surface characteristics, and electrical properties.

### FIELD

The disclosure relates generally to implantable medical devices (“IMDs”) coated using atomic layer deposition (“ALD”) techniques to improve, among other things, hermeticity, biocompatibility, biostability, surface characteristics, and electrical properties.

### BACKGROUND

The implantation of medical devices into the human body for extended periods of time has become a common practice with constantly evolving and expanding applications. Devices designed for long-term use within the body should be designed to minimize the device's undesirable impact on the body environment while at the same time limiting the environment's undesirable impact on the device. Examples of undesirable impact of the device on the body include thrombogenic response, cellular breakdown, and aggravated immune responses. Conversely, the device may be impacted by corrosion and other body fluids, moisture and ionic contaminants that decrease the useful life or performance of the device.

The importance of making implantable medical devices more compatible with the human body becomes significant as device technology enables miniaturization. This is in part because smaller devices require minute components that are susceptible to failure if exposed to otherwise minor bio-contaminants in the body. Further, increased use of media-exposed devices presents new challenges in controlling and possibly eliminating undesireable interactions between an implanted device and the body environment.

### SUMMARY OF THE INVENTION

Embodiments of the invention are generally related to coatings applied to a wide variety of medical devices using atomic layer deposition techniques and methods for applying such coatings. In one embodiment, a feedthrough conductor used to convey electrical signals through the walls of a canister of an implantable medical device, without exposing the interior of the canister to the exterior environment, may be coated using the atomic layer deposition process. In another embodiment media-exposed transducers, possibly installed on an electrical lead, may be coated using the atomic layer deposition process. Embodiments of the present invention also include methods for coating implantable medical devices such as, but not limited to, electrical feedthroughs and media exposed devices with a coating using the atomic layer deposition process.

### DESCRIPTION OF THE DRAWINGS

FIG. 1 is a schematic drawing of an embodiment of an implantable medical device (“IMD”) in accordance with the invention.

FIG. 2 is a cross-section of an embodiment of an electrical feedthrough in accordance with the invention.

FIG. 3 is a flow chart of an embodiment of a process in accordance with the invention.

FIG. 4 is a side view of an exemplary medical lead 4, which may be configured to be coupled to an IMD.

FIG. 5 is a cross-section of an embodiment of an IMD including a transducer in accordance with the invention.

### DETAILED DESCRIPTION

The following detailed description should be read with reference to the drawings, in which like elements in different drawings are numbered identically. The drawings depict selected embodiments and are not intended to limit the scope of the invention. It will be understood that embodiments shown in the drawings and described below are merely for illustrative purposes, and are not intended to limit the scope of the invention as defined in the claims.

Many IMDs include integrated circuits for processing information obtained from sensors (i.e., pressure sensors, electrical activity sensors, etc.) and for administering therapies (i.e., pacing pulses, defibrillation shocks, etc.). In some cases these integrated circuits are housed in hermetically sealed containers or “cans” to minimize undesirable interactions between the integrated circuit and the body. Feedthroughs are used to provide electrical connection between therapy delivery, sensing and detection components (outside the can), while maintaining a hermetic seal at the connection point.

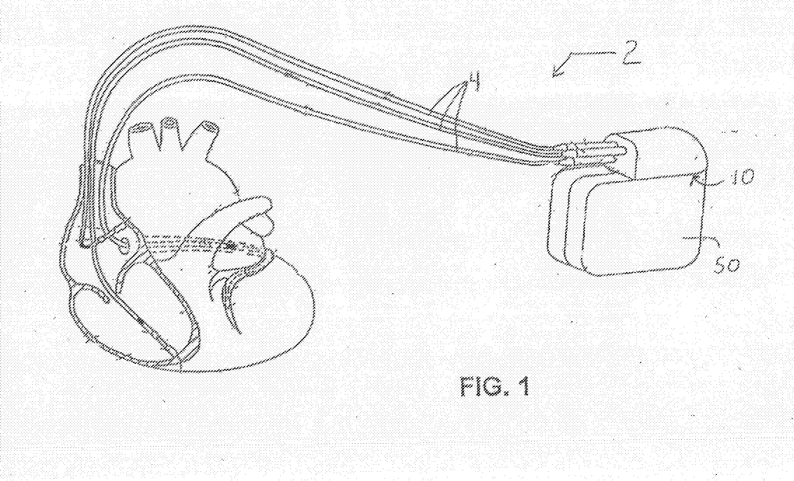

FIG. 1 is a schematic drawing of an embodiment of an IMD in accordance with the invention. The IMD 2 may be used as or employ an implantable cardioverter defibrillator, a cardiac resynchronization therapy device, an implantable hemodynamic monitor, or any other implantable device. The IMD 2 may include leads 4 and a feedthrough generally indicated at 10 (not shown). The feedthrough 10 allows for the electrical connection of the electrical leads 4 to the electronic components inside of the IMD can 50 without compromising the hermeticity at the interface between feedthrough 10 and IMD 2.

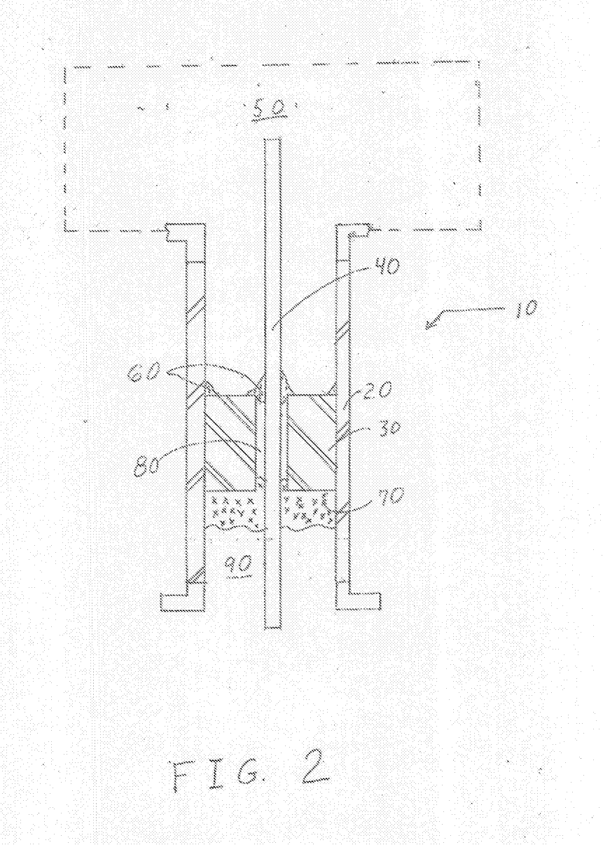

FIG. 2 is a cross-section of an embodiment of an electrical feedthrough in accordance with the invention. Feedthrough 10 includes feedthrough body 20 that houses other elements of the feedthrough 10. An insulating element 30 provides electrical insulation between the feedthrough pin 40 and the feedthrough body 20. The insulating element includes, for example, sapphire, glass, ceramic, polymer, or other material known in the art. The insulating element 30 may be in a disk shape with a hole near the center of the disk for the feedthrough pin 40 to pass through. Other configurations will occur to those of skill in the art upon reading this disclosure.

At the proximal end, the feedthrough pin 40 includes connection pins (not shown) to provide electrical connection with circuits or other controls in IMD can 50. The other end of the feedthrough pin 40 may be connected to a lead (not shown) outside of the IMD can 50. The lead may be connected to a sensor, a therapy delivery device, or other device requiring electrical connection with the contents of the IMD can 50. The feedthrough body 20 may be in electrical communication with the IMD can 50, but the feedthrough pin 40 is isolated from the can by the insulating element 30.

The feedthrough pin 40 may be secured to the insulating element 30 via a brazing process. The braze 60 may be made of a different material of construction than the feedthrough body 20 and/or the feedthrough pin 40. In one exemplary embodiment the feedthrough body 20 is titanium, the feedthrough pin 40 is an alloy of platinum and iridium, and the braze 60 is gold. The braze 60 may wick down the pin 40 and fill most of the annular gap 80. However in some instances bodily fluids may migrate along the feedthrough pin 40 through the annular space 80 around the pin 40. These bodily fluids may corrode the braze joint 60. This problem is more likely to manifest itself in feedthroughs when the electrical current sent through the feedthrough pin 40 is direct current (DC) (i.e., sensors) rather than AC or AC-like as when electrical pulses are intermittently sent through the feedthrough pin 40.

The exterior well 90 of the feedthrough 10 may optionally be filled with a polymer 70. Examples of polymers usable in this application include, but are not limited to, epoxies, polyimides, silicones, and polyurethanes. This polymer fill acts as a hermetic seal against the migration of bodily fluid into the feedthrough 10 and also absorbs any mechanical stress that may act on the feedthrough pin 40 to protect the braze 60 and the insulating element 30. However, even with this polymer barrier, it is possible that additional protection for the feedthrough will be desirable.

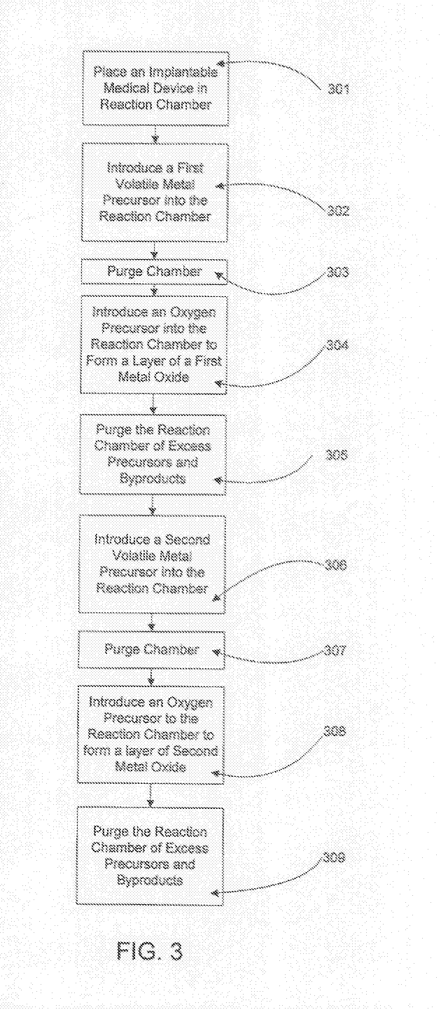

Atomic layer deposition (“ALD”) is a process that allows thin conformal coatings of specific chemistries to be applied to a device or surface in a very controlled fashion. A flow chart of an exemplary ALD process is shown in FIG. 3. ALD is a binary reaction sequence of self-limiting chemical reactions. One atomic layer is deposited during each deposition cycle, so film thickness can be extremely well controlled. An implantable medical device is placed in a reaction chamber 301. A volatile metal precursor is allowed to react with the surface of the implantable medical device 302. For purposes of a non-limiting example, an aluminum precursor could be used to form an aluminum oxide coating. The reaction chamber 301 is then optionally purged 303 and an oxygen precursor is then allowed to react with the metal compound on the surface to form a monomolecular layer of aluminum (metal) oxide 304. The reaction chamber is then purged of excess precursors and reaction byproducts 305 and the process is repeated as necessary to achieve the desired coating thickness. Each layer, first the aluminum, then the reaction to generate the oxide, then the aluminum again, is limited to one atomic layer by the surface controlled chemical reactions. The process for applying other coating material is similar.

It is also possible to create laminated coatings by using different precursors in sequence. For example a layer of aluminum oxide may be followed by a layer of titanium oxide, tantalum pentoxide, or any combination of suitable materials that may be used and will occur to those of ordinary skill in the art. Once an initial layer is created by the execution, perhaps multiple times, of steps 302-305, a second volatile precursor may be introduced into the reaction chamber 306 to react with the previously formed metal oxide. The chamber may be optionally purged 307. An oxygen precursor is now added to the reaction chamber to form a self-limited layer of a second metal oxide 308. Excess precursors and byproducts are then purged from the reaction chamber 309. These steps may be repeated in various sequences to created layered coatings of two or more metal oxides in a very well controlled fashion.

In certain embodiments, an ALD coating of aluminum oxide (Al₂O₃) is applied to a gold brazed feedthrough as described herein. In certain embodiments, a tantalum pentoxide (Ta₂O₅) and a laminate of alternating layers of Al₂O₃ and Ta₂O₅ were also applied. In one case a feedthrough was coated with a 200-nanometer coating of Al₂O₃ and Ta₂O₅. Testing was performed in a saline electrolyte with an electrical differential applied between the feedthrough pin 40 and the feedthrough body 20. The coated feedthrough lasted several months without significant corrosion while the uncoated feedthrough failed in three days. The feedthrough was coated without a sensor or lead attached, but the coating process is amenable to coating the feedthrough itself or the entire device since the process is vapor phase and can coat surfaces that are not “line-of-sight” coatable. That is, because vapor phase coating processes, such as the ALD process, simply allow the coating material to envelope the item to be coated and then the coating is formed on the surface through surface reactions, these processes can coat, for example, the inside surface of an enclosed object through a relatively small opening that can allow access of the vapors to the inside of such an enclosed object. Vapor phase coatings can coat multiple surfaces of an object without the need to rotate the object or move the source of the coating material relative to the object. Other coating processes require a “line of sight,” or a direct line between the source of the coating material and the area of the object to be coated.

The ALD coatings, as just mentioned, are not limited by line of sight. In fact, ALD coatings may have an aspect ratio of 80:1 or more, meaning that a coating may be applied through an opening and coat an area eighty (80) times greater in depth than the effective diameter of the opening itself. This property of ALD coatings enables coating of the interior elements of the IMD 2 through a small opening that may be sealed after the coating process. For example, the interior of an IMD can 50 (and interior components) can be coated effectively through a relatively small opening in the can such as an opening configured to accept a feedthrough. The feedthrough can then be installed in the opening completing the construction of the device.

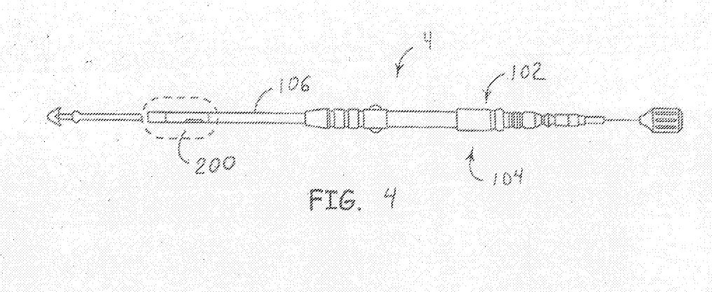

ALD coating techniques, consistent with the present invention, can be used on other implantable devices such as, for example, media-exposed sensors, integrated circuits, micro-electro-mechanical systems, and other media-exposed devices for use with or as an IMD. FIG. 4 is a side view of an exemplary medical lead 4, which is configured to be coupled to an IMD can. The lead 4 includes a transducer module 200. The lead 4 may be any one of a number of different types of leads. For example, the lead 4 may be a pressure monitoring lead, a therapy lead, and other known types of leads, or leads that may be developed in the future that still fall under the appended claims. The lead 4 includes a connector assembly 102, a lead body 106, and transducer module 200. The connector assembly 102 may be located at a proximal section 104 of the lead 100 and may be configured to be coupled to an IMD container to electrically couple the lead 4 thereto.

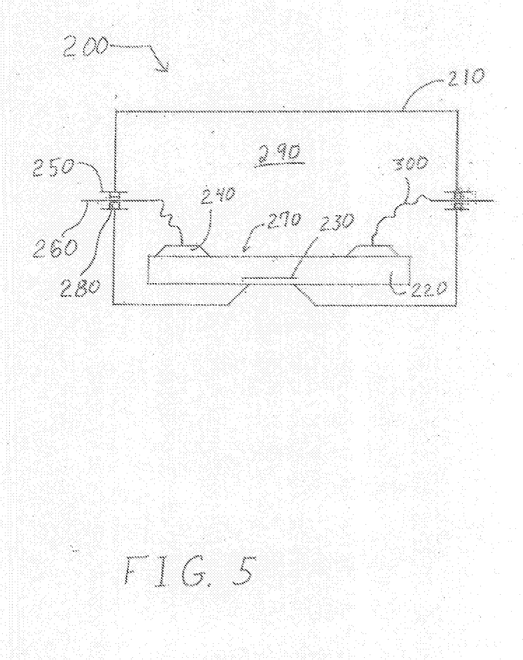

FIG. 5 is a cross-section of an embodiment of an IMD including a transducer in accordance with the invention. The transducer module 200 includes a non-hermetic container or can 210. The container or can 210 may also be hermetically sealed in other embodiments.

Can 210 includes transducer 270. The transducer 270 includes an integrated circuit 220 and sensor 230. The transducer 270 of this embodiment has a sensor, such as a pressure sensor, an optical sensor, a biochemical sensor, a protein sensor, a motion sensor (e.g., an accelerometer or a gyroscope), a temperature sensor, a chemical sensor (e.g., a pH sensor), a genetic sensor, and the like. In some embodiments, the transducer 270 is a sensing transducer, an actuating transducer, an IC-only transducer, or combinations thereof, or other suitable transducers. Examples of sensing transducers include a sensor and an integrated circuit integrated on a chip (e.g., a silicon substrate or micro-electro-mechanical system (MEMS) device or nano-electro-mechanical system (NEMS) device), a sensor element without an integrated circuit built into a substrate (e.g., glass, ceramic, silicon, or other suitable material), and a sensor element built into a substrate with an integrated circuit hermetically encapsulated or packaged into a substrate. Examples of actuating transducers include an actuator and an integrated circuit integrated on a chip (e.g., a silicon substrate or MEMS device or NEMS device), a actuator element without an integrated circuit built into a substrate (e.g., glass, ceramic, silicon, or other suitable material), and an actuator element built into a substrate with an integrated circuit hermetically encapsulated or packaged into a substrate. Such actuating transducers may include a piezoelectric element actuator for vibration. Examples of IC-only transducers include an integrated circuit on a silicon substrate (e.g., an IC-logic multiplexer on a lead or a memory chip for sensor calibration coefficients) and an integrated circuit hermetically encapsulated or packaged into a substrate (e.g., glass, ceramic, silicon, or other suitable material).

In the embodiment shown in FIG. 5, the sensor 230 is a micro-mechanical pressure sensor, but other sensors such as optical sensors, biochemical sensors, protein sensors, motion sensors (e.g., accelerometers or gyroscopes), temperature sensors, chemical sensors (e.g., pH sensors), genetic sensors, electrical activity or others that will occur to those of skill in the art may be employed within the spirit of the invention.

A micro-mechanical pressure sensor that is integrated with the silicon of an integrated circuit 220 may be fabricated by bonding two wafers of silicon together to form a 4-8 μm thick diaphragm or by etching such a diaphragm out of the silicon. Other methods of creating a micro-mechanical pressure sensor are known in the art.

The transducer module 200 of the embodiment shown in FIG. 5 includes feedthroughs 250 having feedthrough pins 260. The feedthroughs 250 are constructed similar to the feedthroughs 10 described above in reference to the embodiments shown in FIG. 2. Since the transducer module 200 may not be hermetically sealed, the feedthroughs 250 are not required to be hermetic. One or both of the feedthrough pins 260 may be electrically isolated from the can 210 by an insulating element 280 as described earlier.

Conductors 300 connect the feedthrough pins 260 to the integrated circuit 220. The conductors 300 electrically connect the feedthrough pins to pads 240 on the integrated circuit 220. The pads 240 may be made of tantalum, niobium, titanium, or other similar electrically conductive material.

In the embodiment shown in FIG. 5, the sensor 230 is media-exposed in that it comes in direct contact with the bodily fluids. In other embodiments, a transducer could be isolated from the media by a membrane and a buffer fluid, wherein the buffer fluid transmits pressure from the membrane to the sensor. In this case, the can 210 may be hermetically sealed to protect the internal components. The exposed pressure sensor 230 of the embodiment of FIG. 5 may use a capacitive or piezoelectric sensor to measure the pressure of the media to which the sensor is exposed. A signal generated by this sensor can then be processed by the integrated circuits and transmitted through the electrical feedthroughs to diagnostic, recording, or other processes.

The inventors have learned that such media-exposed transducers have certain performance characteristics that may be preferable in certain circumstances over transducers that are hermetically sealed. It is also believed that some non-hermetic IMDs are easier and less expensive to construct and maintain and may be more reliable in some applications than hermetically sealed devices. Coating these devices with ALD coatings as described above makes them even more usable as the devices can be protected from the environment and the body can be protected from impacts from the device.

Once the embodiment of the transducer module 200 of FIG. 5 is assembled as just described, an ALD coating may be applied to the interior and exterior of the transducer module 200. The vapor phase nature of ALD coatings permits the interior to be coated through the feedthrough openings and an opening near the sensor 230. Once the transducer module 200 is coated, an optional polymer may be injected to fill the void space 290 in the sensor module 200. Suitable polymers include, but are not limited to, polyimides, polyurethanes, silicones, and epoxies.

The ALD coating in this embodiment may coat the integrated circuit 220, sensor 230, and other components of the transducer module 200 with a thin and conformal coating. This coating can protect the silicon from etching by blood, for example.

Some integrated circuits contain a passivation layer. A passivation layer on the integrated circuit reduces the reactivity of the substrate of the integrated circuit substrate once the circuit has been formed. Such a layer may, for example, be phosphosilicate glass, silicon nitride, polyamide, and combinations thereof. The ALD coating may protect these layers from bodily fluids that may lead to breakdown of the passivation layer. For example, ALD coatings may eliminate or reduce the leaching of bonding agents that are used to connect the glass to the integrated circuit substrate.

Integrated circuits may contain dopants or metals to allow for certain functionality of the integrated circuit. For example, runners or pad out metallization may allow for electrical connectivity within the integrated circuit or between the integrated circuit and other elements or devices. Such runners or metallization may, for example, include metals such as aluminum, aluminum-copper alloy, aluminum-copper-silicon alloy, copper, titanium-tungsten alloy, titanium nitride and others known to those of skill in the art, and this invention contemplates these materials and materials yet to be applied to integrated circuits. As mentioned above, the pads 240 that allow connection of the conductors 300 and feedthrough pins 260 to the transducer 270 may be made of tantalum, niobium, titanium, or other similar electrically conductive material. The ALD coatings may protect these metals from corrosive or other attacks from bodily fluids and prevent these metals from leaching into the body where they may have deleterious effects.

Integrated circuits may also include discrete elements such as capacitors that are soldered onto the integrated circuit. A “lid” may be placed on these integrated circuits for physical protection of the circuit and to provide insulation to prevent short-circuiting among the elements. Often this lid is glass that includes components such as, for example, boron, to help bond the glass to the substrate. ALD coatings on these media-exposed devices may help to prevent the leaching of boron or other dopants from the glass or solder from the discrete elements into the bodily fluids. As the dopant leaches from the glass the glass may lose its dielectric properties. Further, the solder bond may weaken and perhaps fail if enough solder is compromised.

Thus, embodiments atomic layer deposition coatings for implantable medical devices are disclosed. One skilled in the art will appreciate that atomic layer deposition coatings for implantable medical devices can be practiced with embodiments other than those disclosed. Moreover, it should be understood that the features of the disclosed embodiments may be used in a variety of combinations. The combinations of features of the disclosed embodiments are compiled only for purposes of illustration and not limitation. The present invention is limited only by the claims that follow.

1. An implantable medical device, comprising:
a feedthrough conductor including a first metal or alloy that passes from the exterior of a hermetically sealed housing to the interior of the housing; a braze joint comprising a second metal or alloy with a different composition from the first metal or alloy used to secure the feedthrough conductor directly or indirectly to the device; and
a coating formed by atomic layer deposition that covers substantially all of the braze joint.

2. The device of claim 1, wherein the coating is selected from a group consisting of titanium dioxide (TiO₂), aluminum oxide (Al₂O₃), tantalum pentoxide (Ta₂O₅) and layered combinations thereof.

3. The device of claim 1, further comprising a polymer or polymer adhesive at least partially filling a well in the feedthrough conductor located at the exterior of the device.

4. The device of claim 1, wherein the braze joint comprises gold.

5. The device of claim 1, wherein the second metal or alloy has a different galvanic potential than the first metal or alloy.

6. The device of claim 1, wherein the feedthrough conductor is essentially electrically isolated from the device by an insulating element.

7. The device of claim 6, wherein the coating covers substantially all of the insulating element.

8. The device of claim 7, wherein the insulating element is selected from a group consisting of glass, sapphire, polymer, and ceramic.

9. A transducer module of an implantable medical device, comprising:
a transducer; and
a coating formed by atomic layer deposition that covers substantially all of the transducer.

10. The transducer module of claim 9, further comprising a non-hermetic container containing the transducer.

11. The transducer module of claim 10, wherein the container is filled with a polymer that surrounds the transducer.

12. The transducer module of claim 9, further comprising a silicon chip.

13. The transducer module of claim 9, further comprising discreet elements mounted on the chip that are also substantially coated with the coating formed by atomic layer deposition.

14. The transducer module of claim 9, further comprising a passivation layer on the silicon chip.

15. The transducer module of claim 14, wherein the passivation layer is selected from a group consisting of phosphosilicate glass, silicon nitride, silicon oxide, poly-silicon, polyamide, polyimide, parylene, and combinations thereof, wherein the passivation layer is substantially coated with the coating formed by atomic layer deposition.

16. The transducer module of claim 9, further comprising runners or pad out metallization on the silicon chip.

17. The transducer module of claim 16, wherein the metallization is selected from the group consisting of aluminum, aluminum-copper alloy, aluminum-copper-silicon alloy, copper, titanium tungsten alloy, titanium nitride, or combinations thereof.

18. A method of increasing the biocompatibility and biostability of an implantable medical device comprising:
placing an implantable medical device in a reaction chamber;
introducing a first volatile metal precursor that is allowed to react with the surfaces of the implantable medical device into the reaction chamber;
introducing an oxygen precursor to the reaction chamber and allowing the oxygen precursor to react with the metal compound on the surface to form a layer of a first metal oxide; and
purging the reaction chamber of excess precursors and reaction byproducts.

19. The method of claim 18, further comprising:
introducing a second volatile metal precursor that comprises a different metal than the first volatile metal precursor and allowing the second volatile metal precursor to react with the layer of first metal oxide on the implantable medical device;
introducing an oxygen precursor to the reaction chamber and allowing the oxygen precursor to react with the metal compound on the surface to form a layer of second metal oxide comprising a different metal than the metal in the first metal oxide; and
purging the reaction chamber of excess precursors and reaction byproducts.

20. The method of claim 18, wherein the first metal oxide comprises aluminum oxide, titanium dioxide, or tantalum pentoxide.
